(19) United States
(12) Patent Application Publication
(10) Pub. No.: US 2009/0143415 A1
(43) Pub. Date: Jun. 4, 2009

(54) Tetrodotoxin And Its Derivatives For The Treatment Of Central-Nervously Derived Neuropathic Pain

(75) Inventors: Helmut Heinrich Buschmann, Barcelona (ES); Frank Hay Kong Shum, North Vancouver (CA); Kim Noel Fisher, Chestermere (CA); Anh Ho Ngoc, Montreal (CA); Michel Hamon, Paris Cedex (FR)

Correspondence Address: FISH & RICHARDSON PC, P.O. BOX 1022, MINNEAPOLIS, MN, 55440-1022, US

(73) Assignee: Wex Pharmaceuticals, Inc., Vancouver, BC (CA)

(21) Appl. No.: 11/663,268

(22) PCT Filed: Sep. 21, 2005
(86) PCT No.: PCT/EP2005/010213

§ 371 Date: Nov. 4, 2008

Related U.S. Application Data
(60) Provisional application No. 60/611,297, filed on Sep. 21, 2004.

(51) Int. Cl. A61K 31/519; A61P 25/00

(52) U.S. Cl. 514/267; 514/257

(57) ABSTRACT

The present invention refers to the use of a sodium channel blocker such as tetrodotoxin or saxitoxin, their analogues and derivatives as well as their physiologically acceptable salts, in medicinal products for human therapeutics for the treatment of central-nervously derived neuropathic pain.

### FIELD OF THE INVENTION

The present invention refers to the use of a sodium channel blocker such as tetrodotoxin or saxitoxin, their analogues and derivatives as well as their physiologically acceptable salts, in medicinal products for human therapeutics for the treatment of central-nervously derived neuropathic pain.

### BACKGROUND OF THE INVENTION

The treatment of pain conditions is of great importance in medicine. There is currently a world-wide need for additional pain therapy. The pressing requirement for a specific treatment of pain conditions or as well a treatment of specific pain conditions which is right for the patient, which is to be understood as the successful and satisfactory treatment of pain for the patients, is documented in the large number of scientific works which have recently and over the years appeared in the field of applied analgesics or on basic research on nociception.

PAIN is defined by the International Association for the Study of Pain (IASP) as “an unpleasant sensory and emotional experience associated with actual or potential tissue damage, or described in terms of such damage (IASP, Classification of chronic pain, 2^{nd} Edition, IASP Press (2002), 210). Even though pain is always subjective its causes or syndromes can be classified.

Especially neuropathic pain which in the past years has developed into a major health problem in broad areas of the population needs a very specific treatment, especially considering that any treatment of neuropathic pain is extremely sensitive to the causes behind the pain, be it the disease ultimately causing it or the mechanistic pathway over which it develops. So, in a majority of cases a substance being able to treat one subtype of neuropathic pain is not—or is at least not necessarily—able to treat other specific subtypes due to the highly diverse nature of this generalized symptom called neuropathic pain.

Therefore, it was the underlying problem solved by this invention to find new ways of treating neuropathic pain, in this case central-nervously derived neuropathic pain.

So, the main object of this invention is the use of a sodium channel blocker and/or one of its derivatives for the production of a medicament for the treatment of central-nervously derived neuropathic pain. The sodium channel blocker is optionally used in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate.

It was found out that TTX is acting on central-nervously derived neuropathic pain with a surprising and also extremely high potency.

The term “sodium channel blocker” mentioned in this application is defined as a compound that specifically binds to and specifically inhibits sodium channels, which are classified as either TTX-resistant or TTX-sensitive. The term TTX-resistant” and TTX-sensitive refers to a difference in the tightness of TTX binding, with the TTX resistant channel having a binding constant as mentioned in Hunter et al., Current Opinion in CPNS Investigational Drugs 1 (1), 1999 as well as Clare et al. DDT, 5 (11), 2000, 506-520 included here by reference and the TTX sensitive channel having a binding constant as mentioned in Hunter et al., Current Opinion in CPNS Investigational Drugs 1 (1), 1999 as well as Clare et al. DDT, 5 (11), 2000, 506-520. A preferred sodium channel blocker thus binds to a sodium channel with a IC₅₀ of less than 200 □M, preferably less than 100 □M or with an IC₅₀ of 2 □M. Said inhibition refers to suppression or modification of any downstream effect caused by activation of said sodium channels. More preferably, the term “sodium channel blocker” mentioned in this Invention refers to compounds binding to an alpha subunit of sodium channels, especially TTX-resistant or TTX-sensitive sodium channels. More preferably, the term “sodium channel blocker” mentioned in this invention refers to compounds binding to either a SS1 or SS2 region of an alpha subunit of sodium channels, especially TTX-resistant or TTX-sensitive sodium channels. Preferred sodium channel blockers for use in this invention are tetrodotoxin and saxitoxin which both specifically inhibit said sodium channels.

The term “analogues” as used in this application is defined here as meaning a chemical compound that is a derivative of a compound which has similar biochemical activity to that compound. “Analogues” of TTX and STX bind to the same site on the alpha subunit of sodium channels as does TTX and STX.

The term “derivatives” as used In this application is defined here as meaning a chemical compound having undergone a chemical derivation such as substitution or addition of a further chemical group to change (for pharmaceutical use) any of its physico-chemical properties, such as solubility or bioavailability. Derivatives include so-called prodrugs, e.g. ester and ether derivatives of an active compound that yield the active compound per se after administration to a subject.

Examples of well known methods of producing a prodrug of a given acting compound are known to those skilled in the art and can be found e.g. in Krogsgaard-Larsen et al., Textbook of Drugdesign and Discovery, Taylor & Francis (April 2002).

In connection with this invention “neutral form” refers to the non-ionic form but also to (at its isoelectric point) neutrally loaded forms (that means containing an equal amount of positive and negative loads) especially the Zwitter-Ion.

The term “salt” according to this invention is to be understood as meaning any form of the active compound according to the invention in which this compound assumes an ionic form (even in solution) or is charged and—if applicable—is also coupled with a counter-ion (a cation or anion). By this are also to be understood complexes of the active compound with other molecules and ions, in particular complexes which are complexed via ionic interactions. As preferred examples of salts this includes the acetate, mono-trifluoracetate, acetate ester salt, citrate, formate, picrate, hydrobromide, monohydrobromide, monohydrochloride or hydrochloride.

The term “physiologically acceptable salt” in the context of this invention is understood as meaning a “salt” (as defined above) of at least one of the compounds according to the invention which are physiologically tolerated—especially if used in humans and/or mammals.

The term “solvate” according to this invention is to be understood as meaning any form of the active compound according to the invention in which this compound has attached to it via non-covalent binding another molecule (most likely a polar solvent) especially including hydrates and alcoholates, e.g. methanolate.

The term “treatment” or “to treat” in the context of this specification means administration of a compound or formulation according to the invention to prevent, ameliorate or eliminate one or more symptoms associated with central-nervously derived neuropathic pain. Furthermore, the terms “to treat” or “treatment” according to this invention include the treatment of symptoms of central-nervously derived neuropathic pain especially certain subtypes of central-nervously derived neuropathic pain, the treatment of the consequences causing the symptoms, the prevention or the prophylaxis of the symptoms of central-nervously derived neuropathic pain, especially certain subtypes of central-nervously derived neuropathic pain.

The term “ameliorate” in the context of this invention is understood as meaning any improvement on the situation of the patient treated—either subjectively (feeling of or on the patient) or objectively (measured parameters).

“Neuropathic pain” is defined by the IASP as “pain initiated or caused by a primary lesion or dysfunction in the nervous system” (IASP, Classification of chronic pain, 2^{nd} Edition, IASP Press (2002), 210). For the purpose of this invention included under this heading or to be treated as synonymous is “Neurogenic Pain” which is defined by the IASP as “pain initiated or caused by a primary lesion, dysfunction or transitory perturbation in the central or central nervous system”. By the restriction of the use according to the current invention to “central-nervously derived” it is clear that the use is restricted to pain caused or initiated in the central nervous system.

The term “central-nervously derived neuropathic pain” according to this invention is to be understood as meaning a neuropathic pain being initiated or caused by a primary lesion, dysfunction or transitory perturbation in the central nervous system, whereas the “central nervous system” is herewith defined as involving the brain and the spinal cord. An example can be found in Abbadie C., Trends Immunol. 2005 October; 26(19):529-34. The interaction/differences between peripherally- and centrally-derived neuropathic pain regarding the symptoms/signs are explained in detail in Jensen et al., Pain 102 (2003)1-8 and in Klein et al., Pain 15 (2005) 227-233. In a highly preferred use according to the invention the sodium channel blocker is selected from tetrodotoxin or any of its derivatives or analogues and/or saxitoxin or any of its derivatives or analogues, optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate.

In another highly preferred use according to the invention the sodium channel blocker is selected from tetrodotoxin, optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate.

Tetrodotoxin (alternatively in the context of this application abbreviated TTX), also known as Ti Qu Duo Xin, is an alkaloid found in puffer fish (Tetradontiae). The chemical name is Octahydro-12-(Hydroxymethyl)-2-imino-5,9,7,10a-dimethano-10aH-[1,3]dioxocino[6,5-d]pyrimidine-4,7,10,11,12-pentol with a molecular formula C₁₁H₁₇N₃O₈ and a Molecular weight of 319.27. It is a potent non-protein neurotoxin and an indispensable tool for the study of neurobiology and physiology. Tetrodotoxin (TTX) is a marine organic toxin which is mainly found in testicles, ovaries, eggs, livers, spleens, eyeballs, and blood of puffer fish as well as in diverse animal species, including goby fish, newt, frogs and the blue ringed octopus and even in marine alga. Several processes for producing TTX are known.

Usually TTX is extracted from marine organisms (e.g. JP 270719 Goto and Takahashi) but besides numerous others methods of synthesis are also described (and used for the preparation of tetrodotoxin in connection to this invention) in U.S. Pat. No. 6,552,191, U.S. Pat. No. 6,478,966, U.S. Pat. No. 6,562,968 or 2002/0086997, all of which are included here by reference. Tetrodotoxin is a well known compound described for example in WO02/22129 as systemically acting as analgesic. For one of the many descriptions of TTX it is recommended turn to e.g. Tu, Anthony (Ed.) Handbook of Natural Toxins, Vol. 3: Marine Toxins and Venoms, 1988, 185-210 as well as Kao (1966), Pharmacol. Rev. 18:997-1049 and others.

Older journals mention that based on the method described by Tahara in U.S. Pat. No. 1,058,643, there was a product sold in Japan containing a 1% solution of TTX extract for uses such as enuresis (Iwakawa and Kimura, Archiv fuer Experimentelle Pathologie und Pharmakologie (1922), 93, 305-31). There were also trials in the 1930s (Hsiang, Nai Shi; Manshu Igaku Zasshi (1939), 30, 639-47 (German abstr. 179) testing the abilities of TTX for addiction treatment.

Tetrodotoxin is a well known compound described for example in CN 1145225 as acting as an analgesic as well as in the treatment of drug addiction. WO02/22129 describes TTX as systemically acting as an analgesic, including acting on neuropathic pain. This general mentioning of neuropathic pain as an example of pain to be treated with TTX is not dealing with any specific subtype of neuropathic pain, especially not with central-nervously derived neuropathic pain.

The phrase “its (tetrodoxin's) derivatives and analogues” according to this invention is defined—using the definition of U.S. Pat. No. 6,030,974 (included here by reference)—as meaning amino perhydroquinazoline compounds having the molecular formula C₁₁H₁₇N₃O₈. Another definition of “tetrodoxin's derivatives and analogues” according to this invention refers to the definition of U.S. Pat. No. 5,846,975 (included here by reference) as amino hydrogenated quinazolines and derivatives including the substances defined from column 3 line 40 to column 6 line 40. Specifically defined “derivatives and analogues of tetrodotoxin” according to this invention are including but are not limited to anhydro-tetrodotoxin, tetrodaminotoxin, methoxytetrodotoxin, ethoxytetrodotoxin, deoxytetrodotoxin and tetrodonic acid, 6 epi-tetrodotoxin, 11-deoxytetrodotoxin as well as the hemilactal type TTX analogues (e.g. 4-epi-TTX, 6-epi-TTX, 11-deoxy-TTX, 4-epi-11-deoxy-TTX, TTX-8-O-hemisuccinate, chiriquitoxin, 11-nor-TTX-6(S)-ol, 11-nor-TTX-6(R)-ol, 11-nor-TTX-6,6-diol, 11-oxo-TTX and TTX-11-carboxylic acid), the lactone type TTX analogues (e.g. 6-epi-TTX (lactone), 11-deoxy-TTX (lactone), 11-nor-TTX-6(S)-ol (lactone), 11-nor-TTX-6(R)-ol (lactone), 11-nor-TTX-6,6-diol (lactone), 5-deoxy-TTX, 5,11-dideoxy-TTX, 4-epi-5,11-didroxy-TTX, 1-hydroxy-5,11-dideoxy-TTX, 5,6,11-trideoxy-TTX and 4-epi-5,6,11-trideoxy-TTX) and the 4,9-anhydro type TTX analogs (e.g. 4,9-anhydro-TTX, 4,9-anhydro-6-epi-TTX, 4,9-anhydro-11-deoxy-TTX, 4,9-anhydro-TTX-8-O-hemisuccinate, 4,9-anhydro-TTX-11-O-hemisuccinate). The typical analogues of TTX possess only ⅛ to 1/40 of the toxicity of TTX in mice, based upon bioassay in mice. It has been observed that the analogues produce joint action, and do not interact adversely. Examples of TTX analogues include novel TTX analogs isolated from various organisms, as well as those that are partially or totally chemically synthesized (see e.g., Yotsu, M. et al. Agric. Biol. Chem., 53(3):893-895 (1989)). Such analogues bind to the same site on the alpha subunit of sodium channels as does TTX.

According to U.S. Pat. No. 6,030,974,“saxitoxin” or “STX” refers to a compound comprising a tetrahydropurine moiety composed of two guanidine units fused together in a stable azaketal linkage, having a molecular formula CloHI7N704 (mol. wt. 299.30) and to derivatives thereof, including but not limited to hydroxysaxitoxins and neosaxitoxin. Bower et al., Nonprotein Neurotoxins, Clin. Toxicol. 18 (7): 813-863 (1981).

It is to be understood that the use according to the invention is restricted to central-nervously derived neuropathic pain in regards to all the paintypes mentioned in here.

In a highly preferred embodiment of the use according to the invention the central-nervously derived neuropathic pain is central neuropathic pain or central neurogenic pain.

According to the IASP “central neuropathic pain” is defined as “a pain initiated or caused by a primary lesion or dysfunction in the central nervous system” and “central neurogenic pain” is defined as “a pain initiated or caused by a primary lesion, dysfunction or transitory perturbation in the central nervous system” (IASP, Classification of chronic pain, 2^{nd} Edition, IASP Press (2002), 213).

In another preferred embodiment of the use according to the invention the central-nervously derived neuropathic pain is allodynia.

According to the IASP “allodynia” is defined as “a pain due to a stimulus which does not normally provoke pain” (IASP, Classification of chronic pain, 2^{nd} Edition, IASP Press (2002), 210).

In another preferred embodiment of the use according to the invention the central-nervously derived neuropathic pain is causalgia.

According to the IASP “causalgia” is defined as “a syndrome of sustained burning pain, allodynia and hyperpathia after a traumatic nerve lesion, often combined with vasomotor and sudomotor dysfunction and later trophic changes” (IASP, Classification of chronic pain, 2^{nd} Edition, IASP Press (2002), 210).

In another preferred embodiment of the use according to the invention the central-nervously derived neuropathic pain is hyperalgesia.

According to the IASP “hyperalgesia” is defined as “an increased response to a stimulus which is normally painful (IASP, Classification of chronic pain, 2^{nd} Edition, IASP Press (2002), 211).

In another preferred embodiment of the use according to the invention the central-nervously derived neuropathic pain is hyperesthesia.

According to the IASP “hyperesthesia” is defined as “increased sensitivity to stimulation, excluding the senses” (IASP, Classification of chronic pain, 2^{nd} Edition, IASP Press (2002), 211).

In another preferred embodiment of the use according to the invention the central-nervously derived neuropathic pain is hyperpathia.

According to the IASP “hyperpathia” is defined as “a painful syndrome characterized by an abnormally painful reaction to a stimulus, especially a repetitive stimulus, as well as an increased threshold” (IASP, Classification of chronic pain, 2^{nd} Edition, IASP Press (2002), 212).

The IASP draws the following difference between “allodynia”, “hyperalgesia” and “hyperpathia” (IASP, Classification of chronic pain, 2^{nd} Edition, IASP Press (2002), 212):

| | | |
|---|---|---|
| Allodynia | Lowered threshold | Stimulus and response |
| | | mode differ |
| Hyperalgesia | Increased response | Stimulus and response |
| | | rate are the same |
| Hyperpathia | Raised threshold; | Stimulus and response |
| | Increased response | rate may be the same |
| | | or different |

In another preferred embodiment of the use according to the invention the central-nervously derived neuropathic pain is neuralgia.

According to the IASP “neuralgia” is defined as “Pain in the distribution of a nerve or nerves” (IASP, Classification of chronic pain, 2^{nd} Edition, IASP Press (2002), 212).

In another preferred embodiment of the use according to the invention the central-nervously derived neuropathic pain is neuritis.

According to the IASP “neuritis” is defined as “Inflammation of a nerve or nerves” (IASP, Classification of chronic pain, 2^{nd} Edition, IASP Press (2002), 212).

In another preferred embodiment of the use according to the invention the central-nervously derived neuropathic pain is neuropathy.

According to the IASP “neuritis” is defined as “a disturbance of function or pathological change in a nerve: in one nerve mononeuropathy, in several nerves mononeuropthy multiplex, if diffuse and bilateral, polyneuropathy” (IASP, Classification of chronic pain, 2^{nd} Edition, IASP Press (2002), 212).

In human therapeutics, the dose administered is normally between 10 and 4000 μg/day of the sodium channel blocker, especially tetrodotoxin, its derivatives or its analogues, especially the dose of e.g. tetrodotoxin administered is normally between 10 and 4000 μg/day or—given the likely twice per day treatment—between 5 to 2000 μg each given dose, sometimes preferably between 250 and 1000 μg each given dose, sometimes preferably between 25 and 50 μg each given dose depending on the route of administration.

In connection with this invention any amount defined refers to each compound individually not to any combination and refers to the compound having a purity of ≧97%. This on the other hand will exclude any impurity contained within the >3% to be mentioned, defined or referred to as active compound in the sense of this invention. For example this would mean that a formulation containing 0.5 mg tetrodotoxin of 99% purity and 0.8 % anhydrotetrodotoxin will be classified and defined according to this invention as containing just tetrodotoxin as active ingredient.

In a highly preferred embodiment of the invention the use according to the invention the sodium channel blocker, especially the tetrodotoxin, its derivative and/or one of its analogues is used in an amount between 10 μg/day and 4 mg/day.

In a highly preferred embodiment of the invention the used tetrodotoxin, its derivative or its analogue is isolated from a biological source, preferably from fish, especially puffer fish.

In a highly preferred embodiment of the invention the used tetrodotoxin, its derivative or its analogue is synthesized.

Any formulation or pharmaceutical composition according to the invention contains the active ingredient (e.g a sodium channel blocker like TTX (Tetrodotoxin), its derivatives and/or its analogues) as well as optionally at least one auxiliary material and/or additive and/or optionally another active ingredient.

The auxiliary material and/or additive can be specifically selected from conserving agents, emulsifiers and/or carriers for parenteral application. The selection of these auxiliary materials and/or additives and of the amounts to be used depends upon how the pharmaceutical composition is to be applied. Examples include here especially parenteral like intravenous subcutaneous or intramuscular application formulations but which could also be used for other administration routes. The most preferred route is generally systemical, preferably meaning not for local action. Still topical routes are also possible.

Routes of administration of tetrodotoxin its derivatives and its analogues can Include intramuscular injection, intraveneous injection, subcutaneous injection, sublingual, bucal, patch through skin, oral ingestion, implantable osmotic pump, collagen implants, aerosols or suppository.

Included in this invention are especially also methods of treatments of a patient or a mammal, including men, suffering from central-nervously derived neuropathic pain using a sodium channel blocker such as tetrodotoxin or saxitoxin and/or one of its analogues or derivatives optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate,. It is also preferred if the method of treatment is restricted to tetrodotoxin, optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate. It is also preferred if the method of treatment is restricted to tetrodotoxin, in neutral form or as a salt, especially a physiologically acceptable salt, whereas preferably tetrodotoxin, its derivative and/or one of its analogues is used in an amount between 10 μg/day and 4 mg/day, is isolated from a biological source, preferably from fish, especially puffer fish, or is synthesized.

The examples and figures in the following section describing pharmacological trials are merely illustrative and the invention cannot be considered in any way as being restricted to these applications.

Figures:

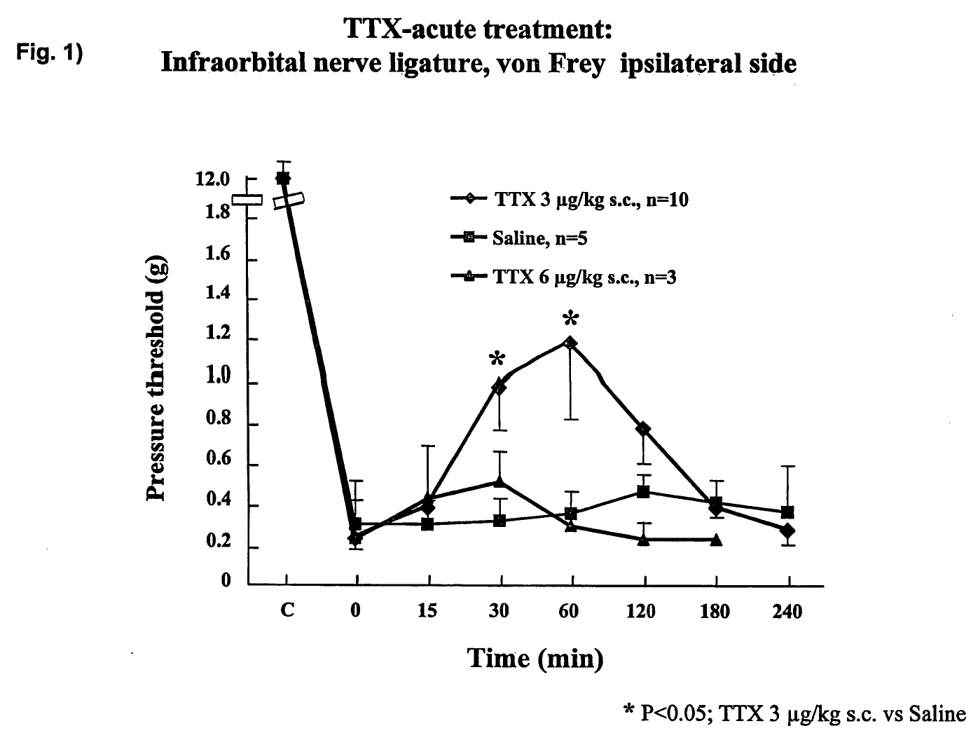

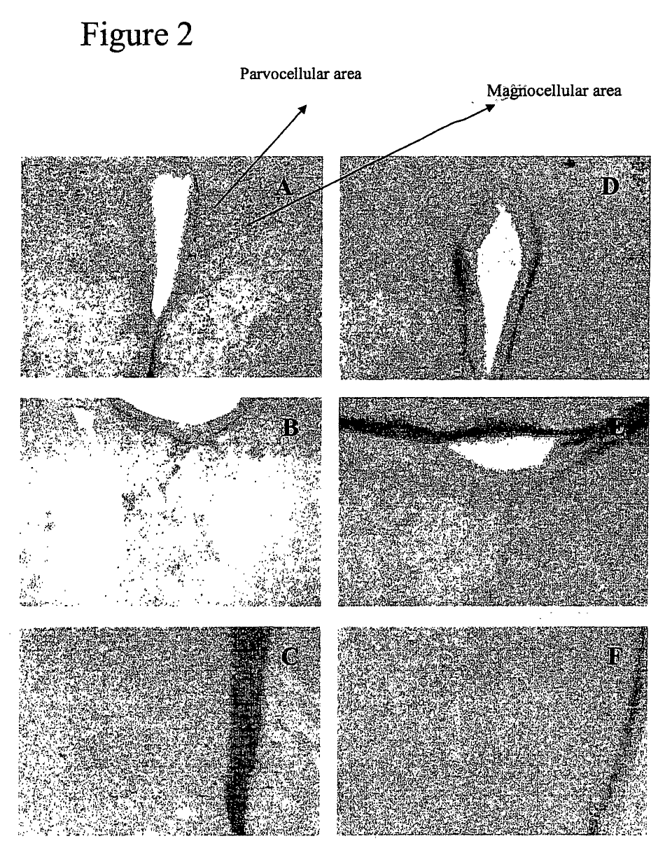

FIG. 1 refers to example 2 and FIG. 2 to example 3.

### EXAMPLES

### Example 1

### Example Formulation of an Injectable (im/iv) Solution of TTX

| | | |
|---|---|---|
| Tetrodotoxin (TTX) (powdered material) | 15 | mg |
| 0.5% diluted acetic acid | 1 | ml |
| Acetic Acid - actetate buffer solution (pH = 3-5) | 50 | ml |
| Water for injection c.s.p., add to | 1000 | ml |

The dosage of TTX for injection is 30 μg in 2 ml.

### Example 2

### Neuropathic Pain—Rats Operated Unilaterally on the Infraorbital Nerve

After pentobarbital-induced anaesthesia, the head of the rat was fixed in a stereotaxic frame, and a mid-line scalp incision was made, exposing skull and nasal bone. The infraorbital part of the right infraorbital nerve was then exposed. The edge of the orbit, formed by the maxillary, frontal, lacrimal and zygomatic bones, was dissected free, and orbital contents were gently deflected to give access to the infraorbital nerve. The latter was then dissected free at its most rostral extent in the orbital cavity, just caudal to the infraorbital foreamen. Two chromic catgut (5-0) ligatures were tied loosely (with about 2 mm spacing) around the nerve (Vos B. P., Strassman A. M. and Maciewitz R. J. (1994) Behavioural evidence of trigeminal neuropathic pain following chronic constriction Injury to rat's infraorbital nerve. J. Neurosci. 14: 2708-2723; and Kayser V., Aubel B., Hamon M. and Bourgoin S. (2002) The antimigraine 5-HT_{1B/1D} receptor agonists, sumatriptan, zolmitriptan and dihydroergotamine, attenuate pain-related behaviour in a rat model of trigeminal neuropathic pain. Br. J. Pharmacol. 137: 1287-1297). Care was taken to avoid any interruption of the epineurial circulation. In sham-operated rats, the right infraorbital nerve was exposed, but it was not ligatured.

In these rats with unilateral ligatures on the infraorbital nerve, sensitivity to mechanical stimulation in the ipsilateral vibrissal territory was assessed by determination of pressure threshold; delivered through von Frey filaments (Semmes-Weinstein monofilaments, Stoelting, Wood Dale, Ill., USA), necessary to trigger defensive behavioural response (brisk withdrawal of the head; attack; escape. reaction). For each stimulus filament (corresponding to a calibrated pressure of 0.217, 0.445, 0.745, 0.976, 2.35, 4.19, 6.00, 7.37 or 12.5 g), three consecutive applications (1 sec apart) were made in order to verify the stability of the response. In both sciatic nerve- and infraorbital nerve- ligatured rats, threshold values were determined first two days before surgery, then 14 days later, at a time when hyperesponsiveness to mechanical and thermal simulations has fully developed (Vos et al., 1994).

Following the operation the response threshold in (g) is reduced considerably if comparing the ipsilateral and contralateral side to the response pre ligature measured as aversive reactions. Tetrodotoxin was given s.c. in a dose of 3 and 6 μg/kg, thus effecting the ipsilateral side results in a strong inducement of the nociceptive threshold (FIG. 1). Saline produced no modification of the nociceptive threshold.

The infraorbital nerve model according to Vos is a very strong model of neuropathic pain, which seems to have some predictability to central nervously derived pain. Especially it remains largely unpredictable, which compounds are active in this model and which not.

### Example 3

### Influence of TTX on c-fos Distribution in the Brain

By immunohistochemistry the effect of TTX (2.5 μg/kg, s.c.) on the expression of the immediate early gene c-Fos, as a marker of neuronal activity was determined. TTX increased c-Fos expression on the paraventricular nuclei of the thalamus and the hypothalamus as well as in the lateral septum (FIG. 2).

TTX was s.c. administered at the dose of 2.5 μg/kg at 10 a.m. Rats were anaesthetised with urethane 90 min after TTX and immediately perfused transcardially with 300 mL saline, followed by 300 mL 4% paraformaldehyde. After perfusion, brains were removed and post-fixed overnight in 4% paraformaldehyde. Coronal sections (40 μm) representative of all the brain and brainstem areas were obtained on a Vibratome (Leica 1000 M). Free-floating sections were bathed in 60% methanol containing 0.3% H₂O₂ for 30 minutes to block endogenous peroxidase activity. Sections were rinsed 3×5 and 1×10 min in 0.1M phosphate buffered saline pH 7.4 (PBS), then 1×10 min in PBS containing 0.1% Triton X-100 (PBS-Triton). Sections were pre-incubated 1×30 min in PBS-Triton containing 5% normal goat serum (PBS-Triton-NS). Anti-c-Fos rabbit antiserum (Calbiochem, USA) was added, at a final dilution of 1:5000 and incubated overnight at 4° C. The next day, sections were washed with PBS (3×5 and 1×10 min) and incubated with goat anti-rabbit secondary antiserum (Vector, USA) diluted in PBS 1:200 for 2 h. Sections were rinsed in PBS (3×5 and 1×10 min) and incubated with the avidin-biotin-peroxidase complex (ABC kit, Vector USA). After washing with 0.05 M Tris-HCl (pH 7.4), sections were developed with 3,3′-diaminobenzidine (Vector, USA), then mounted and coverslipped with DPX (Aldrich, USA). Counting was performed through a 20×air objective by using a Leika DMLS microscope. For each animal, the number of c-Fos stained cells was an average value from 2-3 sections. Cell counts were made randomly by two individuals.

The effect of TTX on c-Fos expression was examined throughout the brain. c-Fos immunostaining in TTX treated rats was not different from that found in control animals in most of the areas examined. Table 1 summarize the effect of TTX in the PVN (F_{(1,9)}=122,302, p<0.001), the PVT (F_{(1,9)}=14,100, p<0.01) and the lateral septum (F_{(1,9)}=36,413, p<0.001). As illustrated in FIG. 2, TTX dramatically enhanced c-Fos immunolabelling in the PVN. A similar result was observed in the PVT as well as in the lateral septum.

TABLE 1
Effect of TTX (2.5 μg/kg) on c-Fos
expression in different brain areas

| | | | Stereotaxic |
|---|---|---|---|
| | Saline | TTX | coordinates |
| Paraventricular | 114.0 ± 29.1 | 703.2 ± 42.1*** | Bregma |
| hypothalamus (PVH) | | | −1.3/−1.8 |
| Paraventricular | 63.8 ± 6.1 | 142.4 ± 18.3** | Bregma |
| thalamus (PVT) | | | −1.3/−1.8 |
| Lateral septum | 18.4 ± 3.8 | 66.2 ± 6.5*** | Bregma |
| | | | 0.7/0.2 |

Each value corresponds to the mean ± S.E.M. of 5-6 data from different animals.
**P < 0.01 and
***P < 0.001 vs saline

In addition to that result it was seen in experiments that TTX can influence the level of some neurotransmitters in the central nervous system.

Accordingly there is a clear proof that TTX is active in the brain showing thus together with the results acquired from the infraorbital nerve model a strong evidence for the activity on centrally-derived neuropathic pain.

1-17. (canceled)

18. A method for manufacture of a composition for treatment of neuropathic pain derived from the central nervous system comprising admixing a sodium channel blocker and/or one of its derivatives, or a solvate, or physiologically acceptable salt thereof, with one or more pharmaceutically acceptable carriers or excipients.

19. The method of claim 18, in which the sodium channel blocker is in the form of a pure stereoisomer, or in the form of a non-racemic mixture of stereoisomers.

20. The method of claim 18, in which the sodium channel blocker is in the form of a neutral salt or neutral compound.

21. The method of claim 18, in which the sodium channel blocker is in the form of a hydrate.

22. The method of claim 18, in which the sodium channel blocker is tetrodotoxin or saxitoxin.

23. The method of claim 21, in which the sodium channel blocker is tetrodotoxin or saxitoxin.

24. The method of claim 18, in which the sodium channel blocker is formulated into a dosage form providing from 10 μg/day to 4 mg/day of the sodium channel blocker.

25. The method of claim 18, wherein the composition is formulated for systemic administration.

26. The method of claim 25, wherein the composition is formulated for oral or parenteral administration.

27. The method of claim 18, wherein the composition is formulated for topical administration.

28. The method of claim 18, in which the pain to be treated is central pain, allodynia, causalgia, hyperalgesia, hyperesthesia, hyperpathia, neuralgia, neuritis or neuropathy.

29. The method of claim 18, wherein the sodium channel blocker is tetrodotoxin, or an analog or derivative thereof, that is isolated from a biological source.

30. The method of claim 18, wherein the sodium channel blocker is synthetic tetrodotoxin, or an analog or derivative thereof.
